(19) United States
(12) Patent Application Publication
(10) Pub. No.: US 2006/0047250 A1
(43) Pub. Date: Mar. 2, 2006

(54) Fluid delivery device

(76) Inventors: Dyson W. Hickingbotham, Stouchsburg, PA (US); Jack R. Prescott, Laguna Hills, CA (US); Heidi A. Prescott, Laguna Hills, CA (US); Michael A. Zica, Costa Mesa, CA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/929,189

(22) Filed: Aug. 30, 2004

(51) Int. Cl. A61M 5/00

(52) U.S. Cl. 604/187

(57) ABSTRACT

A syringe-like device having a chamber in which a pressurizing piston reciprocates. The chamber is connected on one end to a vial containing a drug to be delivered to an eye and on the other end to a needle or cannula for administering the drug to an eye. Pulling proximally backwards on the piston creates a vacuum in the chamber and draws the drug out of the vial. Upon release of the piston, a spring pushes the piston forward, pressurizing the chamber and forcing the drug out of the cannula. A one-way valve prevents the drug from being expelled back into the vial. A pinch valve between the chamber and the cannula allows the flow of the drug out of the cannula to be stopped. The flow rate of the drug out of the cannula can be controlled by varying the size of the chamber, piston and spring.

### BACKGROUND OF THE INVENTION

The present invention generally pertains to the delivery of ophthalmically acceptable pharmaceutically active agents to the back of the eye and more particularly to an apparatus for sub-Tenon delivery of a drug depot to the posterior segment of a human eye proximate the macula.

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, and glaucoma are several examples.

Age related macular degeneration (ARMD) is the leading cause of blindness in the elderly. ARMD attacks the center of vision and blurs it, making reading, driving, and other detailed tasks difficult or impossible. About 200,000 new cases of ARMD occur each year in the United States alone. Current estimates reveal that approximately forty percent of the population over age 75, and approximately twenty percent of the population over age 60, suffer from some degree of macular degeneration. “Wet” ARMD is the type of ARMD that most often causes blindness. In wet ARMD, newly formed choroidal blood vessels (choroidal neovascularization (CNV)) leak fluid and cause progressive damage to the retina.

In the particular case of CNV in ARMD, two main methods of treatment are currently being developed, (a) photocoagulation and (b) the use of angiogenesis inhibitors. However, photocoagulation can be harmful to the retina and is impractical when the CNV is in proximity of the fovea. Furthermore, photocoagulation often results in recurrent CNV over time. Oral administration of anti-angiogenic compounds is also being tested as a systemic treatment for ARMD. However, due to drug-specific metabolic restrictions, systemic administration usually provides sub-therapeutic drug levels to the eye. Therefore, to achieve effective intraocular drug concentrations, either an unacceptably high dose or repetitive conventional doses are required. Various implants have also been developed for delivery of anti-angiogenic compounds locally to the eye. Examples of such implants are disclosed in U.S. Pat. No. 5,824,072 to Wong, U.S. Pat. No. 5,476,511 to Gwon et al., and U.S. Pat. No. 5,773,019 to Ashton et al.

In addition, it is known to use a straight, ⅝ inch long, 25 gauge needle to perform sub-Tenon injection of corticosteroids for the treatment of posterior uveitis or macular edema associated with uveitis or anterior segment surgery. In such methods, a physician attempts to dispose the tip of the needle near the macula but without penetrating the posterior ciliary arteries or the optic nerve. However, because the physician cannot see the tip, as well as movement of the eyeball within the orbit due to contact with the straight needle, it is very difficult to precisely place the tip at the desired location near the macula. For the same reasons, it is also very difficult to determine whether the tip is correctly positioned below the Tenon's capsule. Such methods do not insure a consistent delivery of a specific quantity of drug to a region over the macula. In fact, the literature reports that only about 57 percent of injections using this method result in drug being placed in the sub-Tenon space overlying the macular area. In addition, moving a straight needle along the curved surface of the sclera causes “tenting” or stretching of the overlying Tenon's capsule. Such movement may cause penetration of the Tenon's capsule, allowing drug to be injected into surrounding tissues. Furthermore, such movement may also cause inadvertent penetration of the sclera, resulting in injection of drug into the vitreous cavity. More importantly, penetration of the sclera may result in significant damage to the eye or even a loss of sight. Documented complications of such penetrations include orbital hemorrhage, central retinal vein occlusion, and central retinal artery occlusion.

A further concern with sub-Tenon delivery of a drug depot to the posterior segment of a human eye proximate the macula is that the drug must be administered slowly and under relatively low pressure so as to be retained in the tissue rather than leaking back out of the tissue through the needle channel.

Therefore, a need exists in the field of ophthalmology for an improved apparatus for sub-Tenon delivery of a drug depot to the posterior segment of a human eye proximate the macula.

### BRIEF SUMMARY OF THE INVENTION

The present invention improves upon the prior art by providing a syringe-like device having a chamber in which a pressurizing piston reciprocates. The chamber is connected on one end to a vial containing a drug to be delivered to an eye and on the other end to a needle or cannula for administering the drug to an eye. Pulling proximally backwards on the piston creates a vacuum in the chamber and draws the drug out of the vial. Upon release of the piston, a spring pushes the piston forward, pressurizing the chamber and forcing the drug out of the cannula. A one-way valve prevents the drug from being expelled back into the vial. A pinch valve between the chamber and the cannula allows the flow of the drug out of the cannula to be stopped. The flow rate of the drug out of the cannula can be controlled by varying the size of the chamber, piston and spring.

Accordingly, one objective of the present invention is to provide a syringe-like device having a chamber in which a pressurizing piston reciprocates.

Another objective of the present invention is to provide a device for the sub-Tenon delivery of a drug depot to the posterior segment of a human eye proximate the macula.

Yet another objective of the present invention is to provide a drug delivery device wherein the flow rate of the drug being delivered can be controlled.

These and other advantages and objectives of the present invention will become apparent from the detailed description and claims that follow.

### BRIEF DESCRIPTION OF THE DRAWINGS

FIG. 1 is an enlarged prospective view of the drug deliver device of the present invention.

FIG. 2 is an exploded prospective view of the drug deliver device of the present invention.

FIG. 3 is an enlarged cross-sectional view of the drug deliver device of the present invention.

### DETAILED DESCRIPTION OF THE INVENTION

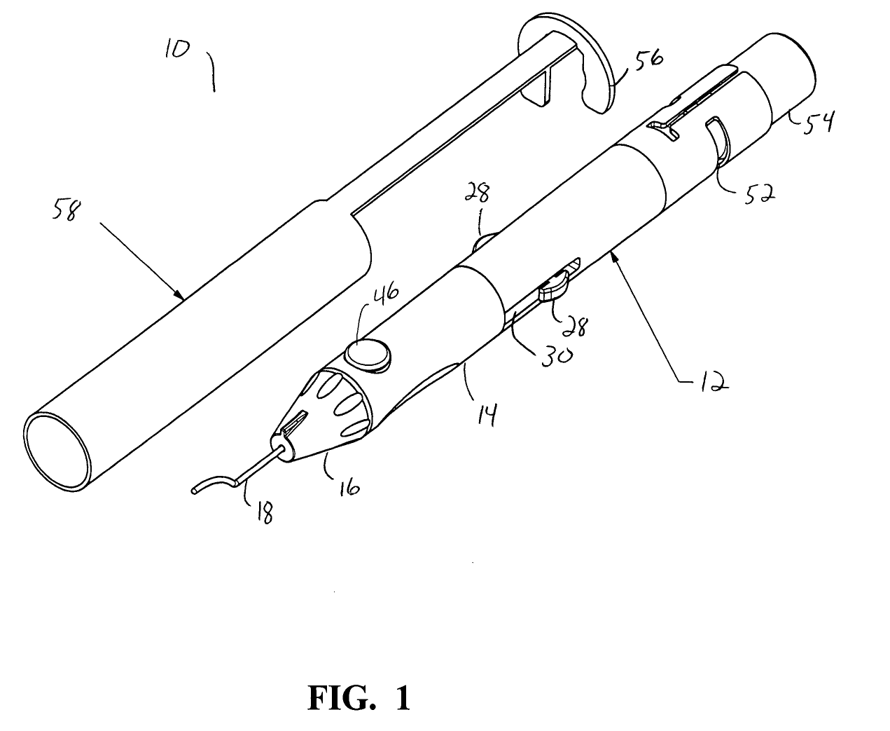

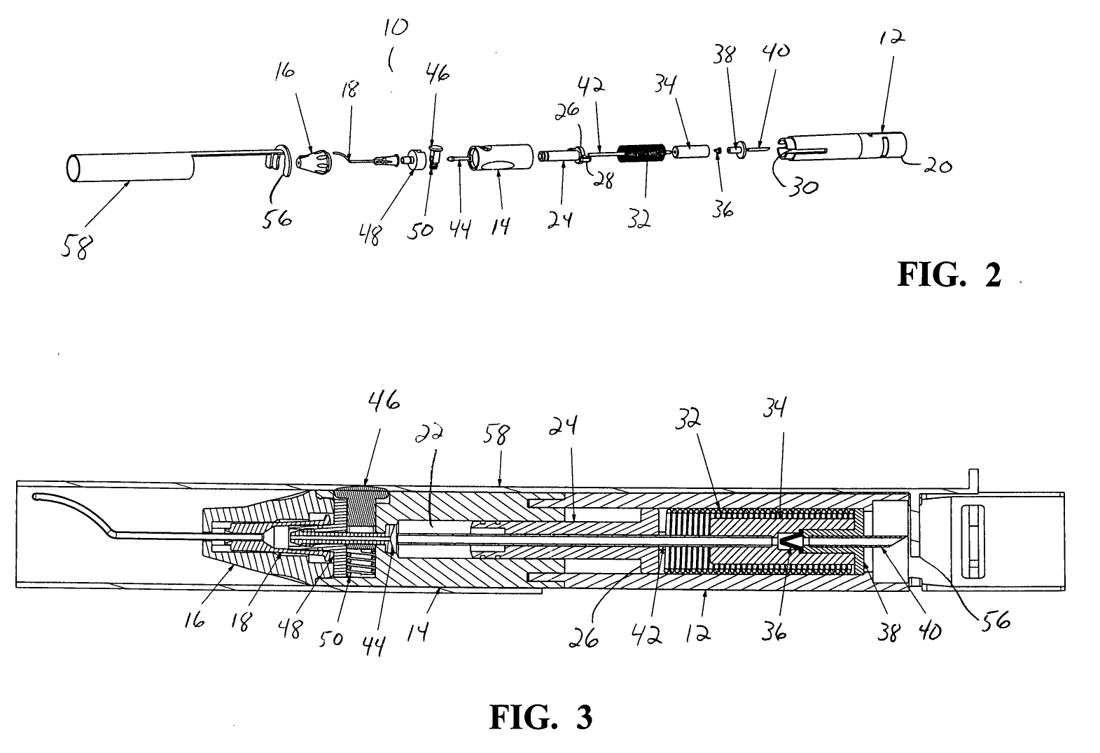

As best seen in FIGS. 1-3, drug delivery device 10 of the present invention generally comprises body 12, cylinder 14, nosecone or tip 16 and cannula 18. Body 12 is generally hollow and proximal end 20 of body 12 is generally open and sized to receive sealed vial 54 of a drug to be delivered. Cylinder 14 likewise is generally hollow having interior chamber 22. Nosecone or tip 16 mounts to cylinder 14 opposite body 12 and retains cannula 18. Cannula 18 may be any suitable cannula, such as the cannula described in U.S. Pat. No. 6,413,245 B1 (Yaacobi, et al.). Piston 24 is generally sized to reciprocate snugly within chamber 22, but contains flange 26 having a plurality of finger tabs 28 that is sized to reciprocate within body 12. Finger tabs 28 fit within slots 30 in body 12 and allow finger tabs 28 to be grasped when piston 24 is installed within device 10. Flange 26 allows piston 24 to engage spring 32, which is mounted inside body 12 between piston 24 and distal end 20. Pulling on finger tabs 28 forces piston 24 to compress spring 32. Coaxially mounted within spring 32 is valve housing 34. Valve housing 34 locates and retains valve 36, valve cap 38 and spike tube 40 within body 12. Extending lengthwise through valve housing 34 and piston 24, from valve 36 to the distal end of chamber 22 is tube 42. Tube 42 provides fluid to be communicated from a vial (not shown) through spike tube 40, valve cap 38 and valve 36 and into chamber 22, as will be described below. Attached to the distal end of cylinder 14, and in fluid communication with chamber 22, is release tubing 44. Release tube 44 communicates with cannula 18 through release button 46 and cannula connector 48. Release button 46 normally pinches shut release tubing 44 and is held in that position by spring 50.

In use, sealed vial 54 containing a drug to be delivered is placed in open proximal end 20 of body 12 so that spike tube 40 pierces into vial 54. During shipment, vial 54 may be held in place within body 12 without being pierced by clips 56 on safety cover 58 being inserted into slots 52 on body 12. Piston 24 is drawn back against spring 32 by grasping and pulling on finger tabs 28. As piston 24 is drawn out of chamber 22, a vacuum is created in chamber 22. The created vacuum draws the drug out of vial 54 through tube 42, valve 36, valve cap 38 and spike tube 40. Once the drug is drawn out of vial 54 and into chamber 22, piston 24 may be released, but piston cannot move forward because chamber 22 is now full of fluid and release button 46 prevents any of the fluid from leaving chamber 22 through release tube 46. Also, valve 36 prevents any of the fluid from flowing back into vial 54. When release button 46 is pressed against spring 50, release tube 44 is unpinched, allowing fluid to flow out of chamber 22 and through cannula 18. The force of spring 32 against piston 24 pressurizes chamber 22, thereby aiding in expressing fluid out of cannula 18.

One skilled in the art will recognize that by varying the size of chamber 22, piston 24 and release tube 44, as well as varying spring 32, specific fluid pressures and flow rates can be achieved

This description is given for purposes of illustration and explanation. It will be apparent to those skilled in the relevant art that changes and modifications may be made to the invention described above without departing from its scope or spirit.

We claim:

1. A fluid delivery device, comprising:
a) a generally hollow, open body sized and shaped to receive a vial of a fluid to be delivered;
b) a cylinder containing a chamber connected to a distal end of the body;
C) a nosecone connected to a distal end of the cylinder and opposite the body, the nosecone retaining a cannula;
d) a spring-loaded piston that reciprocates within the chamber and the body; and
e) a fluid path extending through the body, the cylinder and the nosecone for providing fluid communication from the vial to the cannula.

2. The fluid delivery device of claim 1, wherein the fluid path comprises a spike tube contaned within the body for piercing the vial.

3. The fluid delivery device of claim 1, wherein the fluid path comprises a valve allowing fluid to exit the vial and prevent fluid front entering the vial.

4. The fluid delivery device of claim 1, wherein the fluid path comprises a realease tube for allowing fluid to flow from the chamber to the cannula.

5. The fluid delivery device of claim 4, wherein the fluid path comprises a release tube for preventing the flow of fluid in the release tube.

6. A fluid delivery device, comprising:
a) a generally hollow, open body sized and shaped to receive a vial of a fluid to be delivered;
b) a cylinder containing a chamber connected to a distal end of the body;
c) a nosecone connected to a distal end of the cylinder and opposite the body, the nosecone retaining a cannula;
d) a spring-loaded piston that reciprocates within the chamber and the body; and
e) a fluid path extending through the body, the cylinder and the nosecone for providing fluid communication from the vial to the cannula, the fluid path containing a spike tube for piercing the vial and a release tube for providing fluid communication between the chamber and the cannula.

7. The fluid delivery device of claim 6, wherein the fluid path comprises a release tube for preventing the flow of fluid in the release tube.
